(19) United States
(12) Patent Application Publication
(10) Pub. No.: US 2010/0016382 A1
(43) Pub. Date: Jan. 21, 2010

(54) PHARMACEUTICAL COMPOSITION

(75) Inventors: Yukihiro Nomura, Osaka (JP); Muneo Nonomura, Osaka (JP)

Correspondence Address: WENDEROTH, LIND & PONACK, L.L.P., 1030 15th Street, N.W.,, Suite 400 East, Washington, DC, 20005-1503, US

(73) Assignee: TAKEDA PHARMACEUTICAL COMPANY LIMITED

(21) Appl. No.: 12/310,075

(22) PCT Filed: Aug. 9, 2007
(86) PCT No.: PCT/JP2007/065666

§ 371 Date: Apr. 20, 2009

(30) Foreign Application Priority Data
Oct. 10, 2006 (JP) 2006-218145

(51) Int. Cl. A61K 31/4245; A61P 9/12

(52) U.S. Cl. 514/364

(57) ABSTRACT

The present invention provides a solid pharmaceutical composition superior in the stability and dissolution property, wherein the drug dissolution property of a solid dosage form containing a fat and oil-like substance having a low melting point is improved.

The present invention provides a solid pharmaceutical composition containing an active ingredient, a fat and oil-like substance having a low melting point and a low viscosity binder, and a method of improving dissolution of an active ingredient from a solid pharmaceutical composition containing the active ingredient and a fat and oil-like substance having a low melting point, which includes using a low viscosity binder.

### TECHNICAL FIELD OF THE INVENTION

The present invention relates to a solid pharmaceutical composition superior in stability and dissolution property, which comprises a low viscosity binder.

### BACKGROUND OF THE INVENTION

It is needless to say that pharmaceutical products are required to have effectiveness and safety. To secure effectiveness and safety of a pharmaceutical product, not only the effectiveness and safety of the active ingredient but also the properties from the aspect of manufacturing pharmacy such as stability of the active ingredient in the preparation, dissolution property of the drug from the preparation and the like are extremely important. For example, even if a preparation satisfies a certain level of quality immediately after production, if the active ingredient in the preparation decomposes over time, the preparation is problematic in terms of effectiveness and safety as a pharmaceutical product. As to the dissolution property of the drug from the preparation, when dissolution of the drug from the preparation is too slow, the drug in blood may fail to reach an effective concentration and an expected efficacy may not be achieved. Conversely, when dissolution of the drug from the preparation is too fast, the drug concentration in blood may rapidly increase and the risk of side effects may also increase.

As a method for increasing the stability of the active ingredient in a preparation, addition of a fat and oil-like substance having a low melting point is known. For example, a compound represented by the formula (I) (e.g., benzimidazole-7-carboxylic acid derivative and the like) having a strong angiotensin II receptor antagonistic action and useful as a therapeutic drug for hypertension and the like is a crystalline compound stable to temperature, humidity, heat etc. when it is a single solid compound. However, distortion of crystal due to the pressure, friction, heat and the like applied in granulation or compression during the production process often occurs and decrease in the content with time is accelerated. Decomposition with time of a preparation is known to be suppressed by adding a fat and oil-like substance having a low melting point (patent reference 1: JP-A-5-194218).

On the other hand, in the field of pharmaceutical products, plural preparations containing the same active ingredient at varying drug contents are often sold for the purpose of controlling the dose depending on the severity of the disease and the like. In order to exhibit the efficacy comparable to the content and secure safety in this case, the drug dissolution rate from the preparation needs to be constant irrespective of drug contents. However, it is known that the disintegratability of tablets decreases because tablet weight increases as the scale of tablet increases, and the dissolution property of the drug decreases. Since dissolution of drug from a solid dosage form is correlated with the disintegratability of the solid dosage form, as a method for improving the drug dissolution property from a solid dosage form, the kind and addition method of a disintegrant are generally changed.

### DISCLOSURE OF THE INVENTION

When a fat and oil-like substance having a low melting point was added to improve the stability of the active ingredient in a solid dosage form, disintegratability of the solid dosage form was degraded, the dissolution property of the drug from the solid dosage form decreased markedly. Particularly, degradation of the drug dissolution property was remarkable as the content of the active ingredient in a solid dosage form increased. While the present inventors studied various kinds and addition methods of a disintegrant, the dissolution rate could not be improved.

Therefore, the present inventors have conducted intensive studies in an attempt to improve the drug dissolution property of a solid dosage form containing a fat and oil-like substance having a low melting point and found that the drug dissolution property from the solid dosage form can be unexpectedly improved by the addition of a low viscosity binder to a preparation, which resulted in the completion of the present invention.

Accordingly, the present invention relates to
(1) a solid pharmaceutical composition comprising an active ingredient, a fat and oil-like substance having a low melting point and a low viscosity binder;
(2) the composition of the aforementioned (1), wherein the active ingredient is a crystalline poorly-soluble compound;
(3) the composition of the aforementioned (2), wherein the crystalline poorly-soluble compound has a melting point of about 75° C.- about 250° C., and water solubility of not more than about 1 g/L;
(4) the composition of the aforementioned (2), wherein the crystalline poorly-soluble compound is a compound represented by the formula (I):

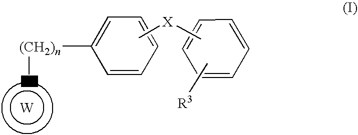

wherein the ring W is an optionally substituted N-containing heterocyclic residue; R³ is a group capable of forming anion or a group convertible thereto; X is a direct bond or a spacer having an atomic length of two or less between the phenylene group and the phenyl group; and n is an integer of 1 or 2, or a salt thereof (hereinafter sometimes to be abbreviated as compound (I));
(5) the composition of the aforementioned (4), wherein the compound represented by the formula (I) or a salt thereof is 2-ethoxy-1-{[2′-(5-oxo-4,5-dihydro-1,2,4-oxadiazol-3-yl)biphenyl-4-yl]methyl}-1H-benzimidazole-7-carboxylic acid (hereinafter sometimes to be abbreviated as compound A);
(6) the composition of the aforementioned (1), wherein the fat and oil-like substance has a melting point of 20° C.-90° C.;
(7) the composition of the aforementioned (1), wherein the low viscosity binder is a low viscosity cellulose derivative;
(8) the composition of the aforementioned (7), wherein the cellulose derivative is hydroxypropylcellulose;
(9) the composition of the aforementioned (1), wherein the low viscosity binder is hydroxypropylcellulose having a viscosity of about 1-about 4 mPa·s;
(10) the composition of the aforementioned (1), which is a tablet;
(11) a method of improving dissolution of an active ingredient from a solid pharmaceutical composition comprising the active ingredient and a fat and oil-like substance having a low melting point, which comprises using a low viscosity binder; and the like.

### BRIEF DESCRIPTION OF THE DRAWINGS

FIG. 1 is a graph showing the dissolution ratios of the Example and Reference Examples.

FIG. 2 is a graph showing the dissolution ratios of the Example and Reference Examples.

The active ingredient in the present invention may be any compound as long as it is a compound whose stability in a solid pharmaceutical composition improves by the addition of a fat and oil-like substance having a low melting point, more specifically, a compound whose physicochemical properties change over time in a solid pharmaceutical composition, wherein the change is suppressed by the addition of a fat and oil-like substance having a low melting point. The properties of the active ingredient in the present invention may be any of solid and fat and oil, preferably solid. When the active ingredient in the present invention is a solid, it may be any of crystal and amorphous, preferably crystal.

Examples of the changes in the physicochemical properties of the active ingredient in a solid pharmaceutical composition include change in the crystallinity degree, change from a certain crystal system to other crystal system, change from anhydride to hydrate, change from hydrate to anhydride, change in the number of hydrate, change from salt to free form, change from free form to salt, change of salt, changes in the chemical structure such as decomposition, oxidation, reduction, polymerization, isomerization and the like, and the like.

As the active ingredient in the present invention, a crystalline poorly-soluble compound is preferable, and a crystalline compound having a melting point of about 75- about 250° C., particularly about 100-about 200° C., is preferable. The “poorly-soluble” means that the solubility in water at 20° C. is specifically not more than about 1 g/L, and a crystalline compound whose solubility in water at 20° C. is preferably not more than about 0.7 g/L, more preferably not more than about 0.5 g/L, is used. While the lower limit of the solubility is not particularly limited, the solubility in water at 20° C. is preferably not less than about 0.001 g/L.

As a crystalline poorly-soluble compound to be used as the active ingredient in the present invention, a compound represented by the formula (I) can be used.

Examples of a group capable of forming an anion (a group having a hydrogen atom capable of being protonated) and a group convertible thereto represented by R³ in the formula (I) include an optionally substituted 5- to 7-membered (preferably 5- to 6-membered) monocyclic heterocyclic residue containing one or more of N, S and O, (for example, tetrazolyl, a group represented by the formula:

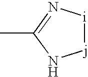

wherein i is —O— or —S—, j is >C═O, >C═S or >S(O)m wherein m is 0, 1 or 2 (e.g., a 5-oxo-4,5-dihydro-1,2,4-oxadiazol-3-yl group and the like) and the like), carboxyl, trifluoromethanesulfonylamino, phosphono, sulfo, cyano, lower (C₁₋₄) alkoxy-carbonyl and the like, and a group convertible to these in the body. Such groups are optionally protected with an optionally substituted lower alkyl group, an acyl group etc., and may include those capable of forming anions or convertible thereto chemically or under biological, i.e., physiological conditions (for example, in vivo reaction and the like, such as oxidation, reduction or hydrolysis catalyzed by in vivo enzymes and the like). Other examples of R³ include those simultaneously having an amino group or a hydroxyl group as a proton donor and a carbonyl group, a thiocarbonyl group or a sulfinyl group as a proton acceptor (e.g., oxadiazolyl, thiadiazolyl and the like).

The 5-oxo-4,5-dihydro-1,2,4-oxadiazol-3-yl group contains three tautomers (a′, b′ and c′) represented by the formulas:

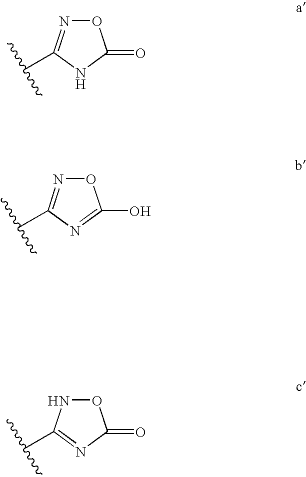

and the 5-oxo-4,5-dihydro-1,2,4-oxadiazol-3-yl group contains all of the above-mentioned a′, b′ and c′.

R³is preferably a tetrazolyl group, a group represented by the formula:

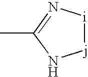

wherein each symbol is as defined above, (e.g., a 5-oxo-4,5-dihydro-1,2,4-oxadiazol-3-yl group and the like), a carboxyl group and the like each optionally protected with an optionally substituted lower (C₁₋₄) alkyl group (e.g. methyl, triphenylmethyl, methoxymethyl, ethoxyethyl, p-methoxybenzyl, p-nitrobenzyl, etc.) or an acyl group (e.g. a lower (C₂₋₅) alkanoyl, benzoyl, etc.), particularly preferably a 5-oxo-4,5-dihydro-1,2,4-oxadiazol-3-yl group.

The substitution position of R³ may be any of the ortho, meta and para positions, with preference given to the ortho position.

X shows that the adjacent phenylene group and phenyl group are bonded to each other directly or via a spacer having not more than 2 atomic chains (preferably a direct bond). As the spacer, any can be used as long as it is a divalent chain in which the number of atoms constituting the straight chain is 1 or 2, and the spacer may have a side chain. Specifically, as the spacer, lower (C₁₋₂) alkylene, —CO—, —O—, —S—, —NH—, —CO—NH—, —O—CH₂—, —S—CH₂—, —CH═CH— and the like can be mentioned.

n is an integer of 1 or 2 (preferably 1).

The group represented by the formula:

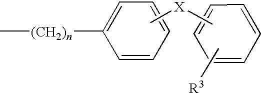

wherein each symbol is as defined above, is preferably a group represented by the formula:

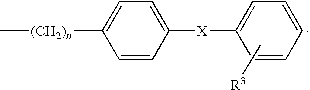

Representative examples of the nitrogen-containing heterocyclic residue represented by the ring W include, but are not limited to, the residue represented by the below-mentioned formula (III) and formula (IV). Specific examples are shown below. In the following formulas, R¹ is a hydrogen atom or an optionally substituted hydrocarbon residue; and Y is a bond, —O—, —S(O)m- (where m is 0, 1 or 2) or —N(R⁴)— (where R⁴ is a hydrogen atom or an optionally substituted alkyl group). Particularly, R¹ is preferably a lower (C₁₋₅) alkyl (preferably a lower (C₂₋₃) alkyl) optionally substituted by a hydroxyl group, an amino group, a halogen atom or a lower (C₁₋₄) alkoxy group; and Y is preferably a bond, —O—, —S— or —N(R⁴)— (wherein R⁴ is a hydrogen atom or a lower (C₁₋₄) alkyl). Examples of the residue represented by the formula (III):

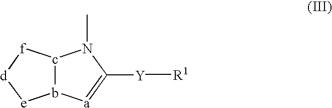

wherein a and e constituting the heterocyclic residue are each independently one or two carbon or hetero atoms each substituted optionally; d and f constituting the heterocyclic residue are each independently an optionally substituted carbon or hetero atom; and b and c constituting the heterocyclic residue are each independently an optionally substituted carbon or nitrogen atom, include the formulas:

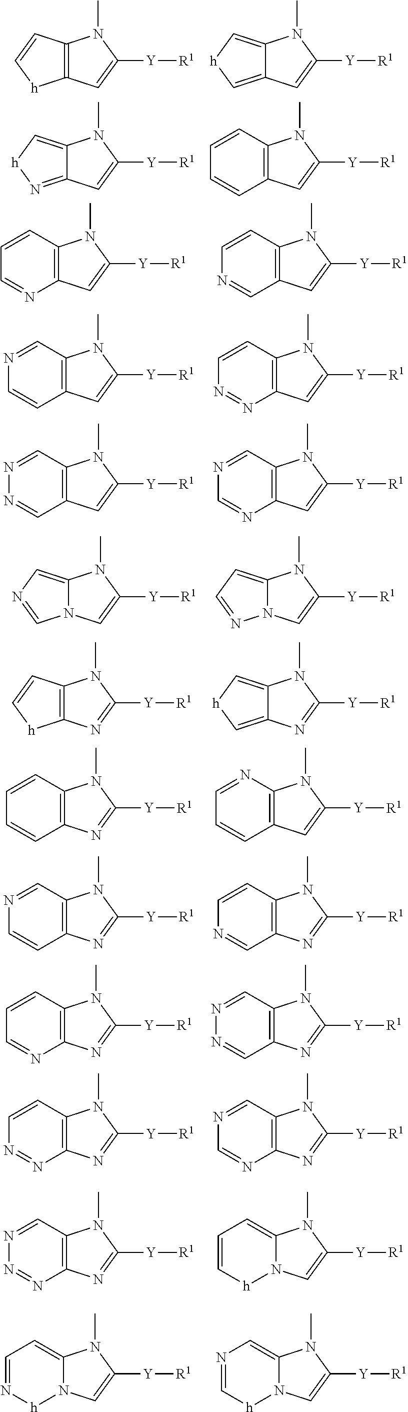

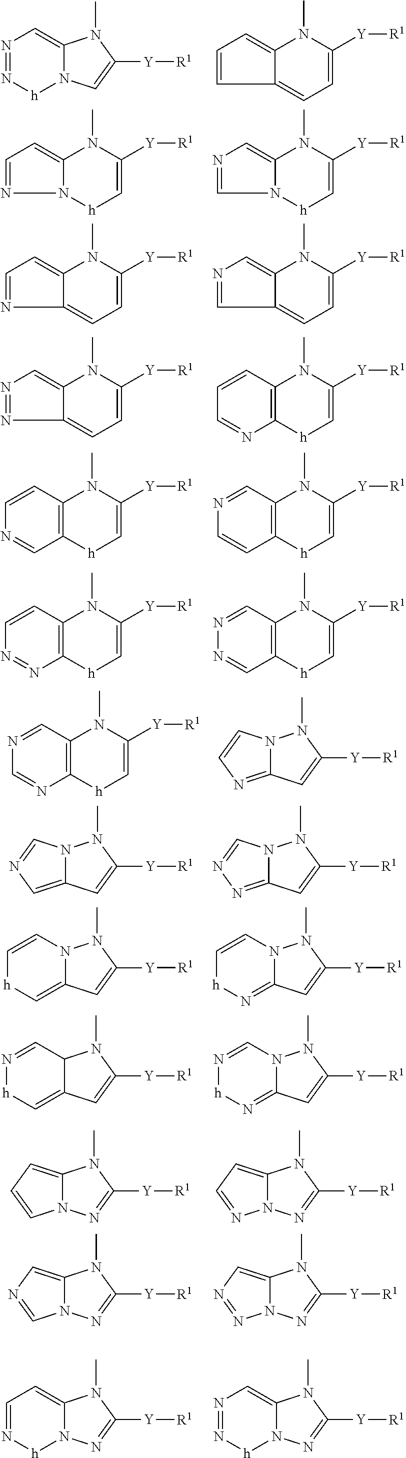

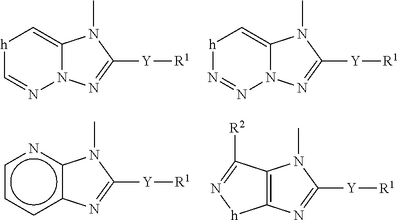

wherein h is >CH₂, >═O, >═S, >S—(O)m, —N(R⁴)— or —O—; m is 0, 1 or 2 and R⁴ is a hydrogen atom or an optionally substituted lower alkyl group (preferably a hydrogen atom or lower (C₁₋₄) alkyl), and the like. Examples of the residue represented by the formula (IV):

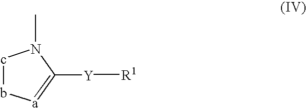

wherein a constituting the heterocyclic residue shows one or two carbon or nitrogen atoms each substituted optionally, b constituting the heterocyclic residue shows one or two carbon or hetero atoms each substituted optionally, and c constituting the heterocyclic residue shows an optionally substituted carbon or hetero atom, include the formulas:

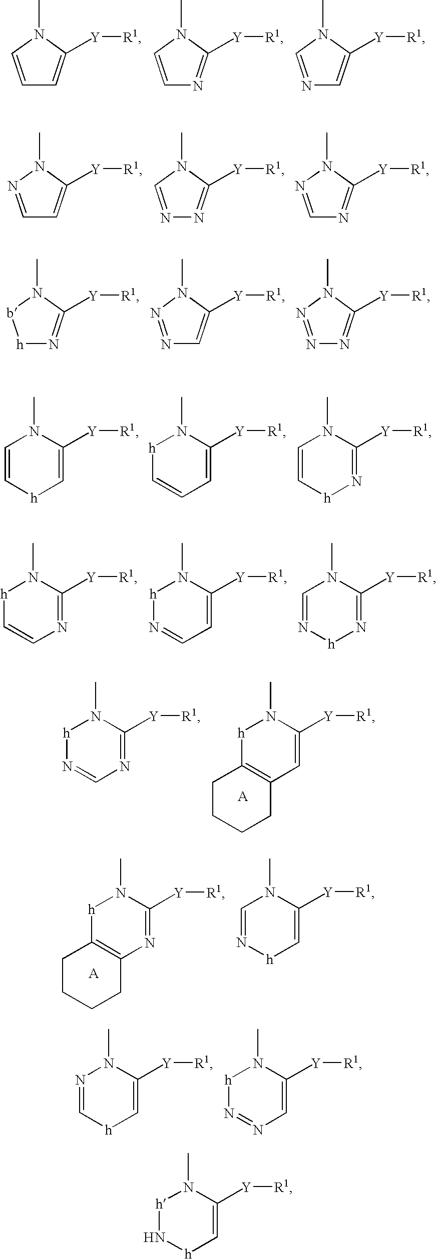

wherein A is an optionally substituted aromatic hydrocarbon residue optionally containing heteroatom, or a heterocyclic residue (preferably an aromatic hydrocarbon residue such as phenyl), h and h′ are each >CH₂, >═O, >═S, >S—(O)ₘ, —N(R⁴)— or —O—, and m and R⁴ are as defined above and the like. The heterocyclic residue represented by the above-mentioned formula (III) is optionally substituted, besides the group represented by Y—R¹, by a group represented by R² (e.g. a group capable of forming an anion or a group convertible thereto). The substitutable position of R² is preferably the position of f in the formula (III).

Examples of the group capable of forming anion or convertible thereto for R² include optionally esterified or amidated carboxyl, tetrazolyl, trifluoromethanesulfonylamino (—NHSO₂ CF₃), phosphono, sulfo and the like. These groups are optionally protected by an optionally substituted lower alkyl group, acyl group and the like, and may be any as long as they are capable of forming anion chemically or under biological i.e., physiological conditions (for example, an in vivo reaction and the like such as oxidation, reduction, hydrolysis etc. by enzymes etc. in the body).

Examples of the optionally esterified or amidated carboxyl for R² include groups represented by the formula: —CO— D [wherein D is hydroxyl group, optionally substituted amino (e.g. amino, N-lower (C₁₋₄) alkylamino, N,N-di-lower (C₁₋₄) alkylamino etc.) or optionally substituted alkoxy {e.g. a lower (C₁₋₆) alkoxy group wherein the alkyl moiety is optionally substituted by a hydroxyl group, an optionally substituted amino (e.g. amino, dimethylamino, diethylamino, piperidino, morpholino etc.), halogen, lower (C₁₋₆) alkoxy, lower (C₁₋₆) alkylthio or optionally substituted dioxolenyl (e.g. 5-methyl-2-oxo-1,3-dioxolen-4-yl etc.), or a group represented by the formula: —O—CH(R⁶)—OCOR₅ [wherein R⁶ is a hydrogen atom, a straight-chain or branched lower (C₁₋₆) alkyl group (e.g. methyl, ethyl, n-propyl, isopropyl, n-butyl, isobutyl, t-butyl, n-pentyl, isopentyl, neopentyl etc.), a straight-chain or branched lower (C₂₋₆) alkenyl group or a C₃₋₈ cycloalkyl group (e.g. cyclopentyl, cyclohexyl, cycloheptyl etc.), and R⁵ is a straight-chain or branched lower (C₁₋₆) alkyl group (e.g. methyl, ethyl, n-propyl, isopropyl, n-butyl, isobutyl, sec-butyl, t-butyl, n-pentyl, isopentyl, neopentyl etc.), a straight-chain or branched lower (C₂₋₆) alkenyl group, a C₃₋₈ cycloalkyl group (e.g. cyclopentyl, cyclohexyl, cycloheptyl etc.), a lower (C₁₋₃) alkyl group substituted by C₃₋₈ cycloalkyl (e.g. cyclopentyl, cyclohexyl, cycloheptyl etc.) or an optionally substituted aryl group such as phenyl (e.g. benzyl, p-chlorobenzyl, phenethyl, cyclopentylmethyl, cyclohexylmethyl etc.), a lower (C₂₋₃) alkenyl group optionally substituted by C₃₋₈ cycloalkyl or an optionally substituted aryl group such as phenyl (e.g. cinnamyl, etc. having alkenyl moiety such as vinyl, propenyl, allyl, isopropenyl etc.), an optionally substituted aryl group such as phenyl (e.g. phenyl, p-tolyl, naphthyl etc.), a straight-chain or branched lower (C₁₋₆) alkoxy group (e.g. methoxy, ethoxy, n-propoxy, isopropoxy, n-butoxy, isobutoxy, sec-butoxy, t-butoxy, n-pentyloxy, isopentyloxy, neopentyloxy etc.), a straight-chain or branched lower (C₂₋₈) alkenyloxy group (e.g. allyloxy, isobutenyloxy etc.), a C₃₋₈ cycloalkyloxy group (e.g. cyclopentyloxy, cyclohexyloxy, cycloheptyloxy etc.), a lower (C₁₋₃) alkoxy group substituted by C₃₋₈ cycloalkyl (e.g. cyclopentyl, cyclohexyl, cyloheptyl etc.) or an optionally substituted aryl group such as phenyl (e.g. benzyloxy, phenethyloxy, cyclopentylmethyloxy, cyclohexylmethyloxy etc., having alkoxy moiety such as methoxy, ethoxy, n-propoxy, isopropoxy etc.), a lower (C₂₋₃) alkenyloxy group substituted by C₃₋₈ cycloalkyl (e.g. cyclopentyl, cyclohexyl, cycloheptyl etc.) or an optionally substituted aryl group such as phenyl (e.g. cinnamyloxy having an alkenyloxy moiety such as vinyloxy, propenyloxy, allyloxy, isopropenyloxy etc.) and an optionally substituted aryloxy group such as phenoxy (e.g. phenoxy, p-nitrophenoxy, naphthoxy etc.) and the like] and the like}] and the like.

Examples of the substituent for R² include a group capable of forming anion or a group convertible thereto (e.g. tetrazolyl, carboxyl, trifluoromethanesulfonylamino, phosphono, sulfo and the like, each optionally protected with alkyl (e.g. a lower (C₁₋₄) alkyl etc.) or acyl (e.g. lower (C₂₋₅) alkanoyl, optionally substituted benzoyl etc.).

The group capable of forming anion or convertible thereto may be any as long as it is capable of forming anion (e.g., COO⁻, a derivative thereof and the like) or convertible thereto chemically or under biological i.e., physiological conditions (for example, an in vivo reaction such as oxidation, reduction, hydrolysis etc. by enzymes in the body). R² may be a carboxyl group, or a prodrug thereof. R² may be biologically or chemically converted to anion in the living body and the like.

Examples of the substituent R² include —COOH and a salt thereof, —COOMe, —COOEt, —COOtBu, —COOPr, pivaloyloxymethoxycarbonyl, 1-(cyclohexyloxycarbonyloxy)ethoxycarbonyl, 5-methyl-2-oxo-1,3-dioxolen-4-ylmethoxycarbonyl, acetoxymethyloxycarbonyl, propionyloxymethoxycarbonyl, n-butyryloxymethoxycarbonyl, isobutyryloxymethoxycarbonyl, 1-(ethoxycarbonyloxy)ethoxycarbonyl, 1-(acetyloxy)ethoxycarbonyl, 1-(isobutyryloxy)ethoxycarbonyl, cyclohexylcarbonyloxymethoxycarbonyl, benzoyloxymethoxycarbonyl, cinnamyloxycarbonyl, cyclopentylcarbonyloxymethoxycarbonyl and the like.

R² is preferably a group represented by the formula: —CO-D, wherein D is a hydroxyl group or a lower (C₁₋₄) alkoxy wherein the alkyl moiety is optionally substituted by a hydroxyl group, amino, halogen, lower (C₂₋₆) alkanoyloxy (e.g., acetyloxy, pivaloyloxy, etc.), lower (C₁₋₆) alkoxy-carbonyloxy (e.g. methoxycarbonyloxy, ethoxycarbonyloxy, cyclohexyloxycarbonyloxy, etc.) or a lower (C₁₋₄) alkoxy.

The heterocyclic residue represented by the formula (III) optionally further has, besides the groups represented by Y—R¹ and R², a substituent exemplified by halogen (e.g. F, Cl, Br etc.), cyano, nitro, lower (C₁₋₄) alkyl, lower (C₁₋₄) alkoxy, an optionally substituted amino group [e.g. amino, N-lower (C₁₋₄) alkylamino (e.g. methylamino etc.), N,N-di-lower (C₁₋₄) alkylamino (e.g. dimethylamino etc.), N-arylamino (e.g. phenylamino etc.), alicyclic amino (e.g. morpholino, piperidino, piperazine, N-phenylpiperazino etc.)], a group represented by the formula: —CO-D′, wherein D′ is hydroxyl group or lower (C₁₋₄) alkoxy wherein the alkyl moiety is optionally substituted by a hydroxyl group, lower (C₁₋₄) alkoxy, lower (C₂₋₆) alkanoyloxy (e.g. acetyloxy, pivaloyloxy, etc.) or lower (C₁₋₆) alkoxycarbonyloxy (e.g. methoxycarbonyloxy, ethoxycarbonyloxy, cyclohexyloxycarbonyloxy, etc.) and tetrazolyl, trifluoromethanesulfonylamino, phosphono or sulfo, each optionally protected with lower (C₁₋₄) alkyl or acyl (e.g. lower (C₂₋₅) alkanoyl, optionally substituted benzoyl etc.). One or two of these substituents are optionally substituted simultaneously on optional positions of the ring constituting the heterocyclic residue. As the nitrogen-containing fused heterocyclic residue represented by the formula (III), the formulas:

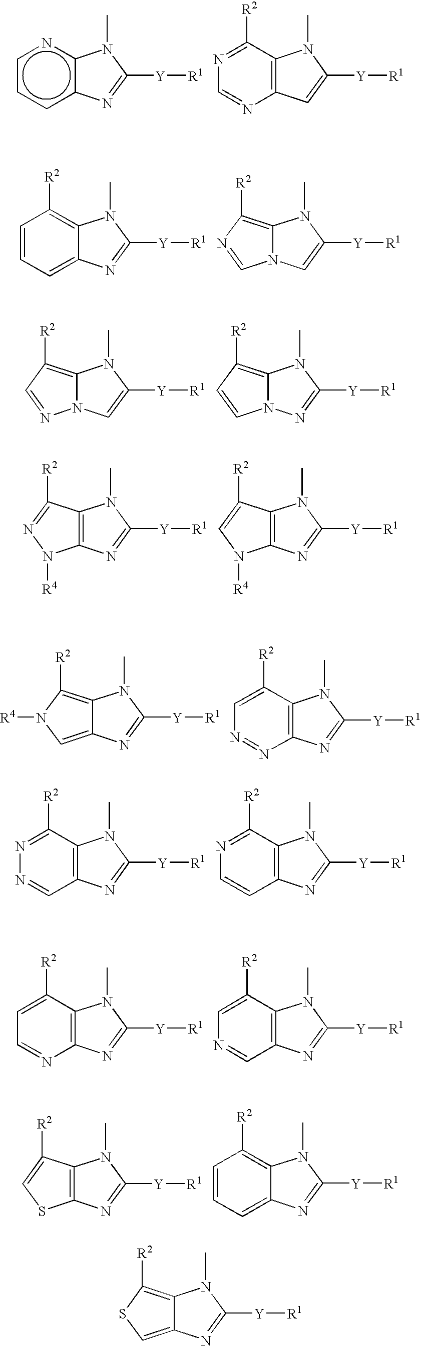

wherein Y—R¹, R² and R⁴ are as defined above, are preferable, and benzimidazolyl, thienoimidazolyl and imidazopyridinyl (particularly benzimidazolyl and thienoimidazolyl) are preferable.

Of the compounds represented by the above-mentioned formula (I), compounds represented by the formula (I′):

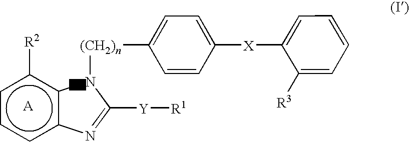

wherein ring A is a benzene ring which may further have a substituent besides the group represented by R²; R¹ is a hydrogen atom or an optionally substituted hydrocarbon residue; R³ is a group capable of forming anion or a group convertible thereto; X shows that phenylene group and phenyl group are bonded directly or via a spacer having not more than two atomic chains; R² is an optionally esterified carboxyl group; Y is a bond, —O—, —S(O)m- (where m is 0, 1 or 2) or —N(R⁴)— (where R⁴ is a hydrogen atom or an optionally substituted alkyl group); and n is an integer of 1 or 2] or salts thereof (hereinafter sometimes to be referred to as compound (I′)). More specifically, of the benzimidazole-7-carboxylic acid or a derivative thereof disclosed in EP-A-0425921 and EP-A-0459136, any crystalline poorly-soluble compound can be employed.

Of these, preferred is compound (I′), which is compound (I′) wherein R¹ is a lower (C₁₋₅) alkyl (preferably a lower (C₂₋₃) alkyl) optionally substituted by a hydroxyl group, an amino group, halogen or a lower (C₁₋₄) alkoxy group; R² is a group represented by the formula: —CO-D, [wherein D is a hydroxyl group or a lower (C₁₋₄) alkoxy wherein the alkyl moiety is optionally substituted by a hydroxyl group, amino, halogen, lower (C₂₋₆) alkanoyloxy (e.g. acetyloxy, pivaloyloxy, etc.), lower (C₁₋₆) alkoxy-carbonyloxy (e.g. methoxycarbonyloxy, ethoxycarbonyloxy, cyclohexyloxycarbonyloxy, etc.) or lower (C₁₋₄) alkoxy]; ring A is a benzene ring which may further have, besides the group represented by R², substituents selected from halogen (e.g. F, Cl, Br, etc.), lower (C₁₋₄) alkyl, lower (C₁₋₄) alkoxy, nitro, a group represented by the formula: —CO-D′ [wherein D′ is a hydroxyl group or lower (C₁₋₄) alkoxy wherein the alkyl moiety is optionally substituted by a hydroxyl group, lower (C₁₋₄) alkoxy, lower (C₂₋₆) alkanoyloxy (e.g. acetyloxy, pivaloyloxy, etc.) or lower (C₁₋₆) alkoxy-carbonyloxy (e.g. methoxycarbonyloxy, ethoxycarbonyloxy, cyclohexyloxycarbonyloxy, etc.)], and amino optionally substituted by lower (C₁₋₄) alkyl, preferably a benzene ring optionally having a substituent such as lower (C₁₋₄) alkyl, halogen etc. other than a group for R², more preferably a benzene ring having no substituents other than a group for R²; Y is a bond, —O—, —S— or N(R⁴)— [wherein R⁴ is a hydrogen atom or lower (C₁₋₄) alkyl]: R³ is a tetrazolyl group, a compound represented by the formula:

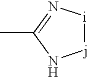

wherein i is —O— or —S—, j is >C═O, >C═S or >S(O)m (wherein m is 0, 1 or 2), (e.g., a 5-oxo-4,5-dihydro-1,2,4-oxadiazol-3-yl group and the like) or a carboxyl group, each of which is optionally protected with optionally substituted lower (C₁₋₄) alkyl (e.g. methyl, triphenylmethyl, methoxymethyl, ethoxymethyl, p-methoxybenzyl, p-nitrobenzyl, etc.) or an acyl group (lower (C₂₋₅) alkanoyl, benzoyl, etc.); n is 1; and X is a bond.

As the compound represented by the formula (I), 2-ethoxy-1-{[2′-(5-oxo-4,5-dihydro-1,2,4-oxadiazol-3-yl)biphenyl-4-yl]methyl}-1H-benzimidazole-7-carboxylic acid, 2-ethoxy-1-{[2′-(1H-tetrazol-5-yl)biphenyl-4-yl]methyl}benzimidazole-7-carboxylic acid (candesartan) or 1-(cyclohexyloxycarbonyloxy)ethyl 2-ethoxy-1-{[2′-(1H-tetrazol-5-yl)biphenyl-4-yl]methyl}benzimidazole-7-carboxylate (candesartan cilexetil) is preferably used, and particularly, 2-ethoxy-1-{[2′-(5-oxo-4,5-dihydro-1,2,4-oxadiazol-3-yl)biphenyl-4-yl]methyl}-1H-benzimidazole-7-carboxylic acid is preferably used.

As salts of the compound represented by the formula (I), pharmaceutically acceptable salts can be mentioned and, for example, salts of a compound represented by the formula (I) with inorganic base, salts thereof with organic base, salts thereof with inorganic acid, salts thereof with organic acid, salts thereof with basic or acidic amino acid and the like can be mentioned. As preferable examples of the salts with inorganic base, for example, alkali metal salts such as sodium salt, potassium salt and the like; alkaline earth metal salts such as calcium salt, magnesium salt and the like; aluminum salt, ammonium salt and the like can be mentioned. As preferable examples of salts with the organic base, for example, salts with trimethylamine, triethylamine, pyridine, picoline, ethanolamine, diethanolamine, triethanolamine, dicyclohexylamine, N,N′-dibenzylethylenediamine and the like can be mentioned. As preferable examples of the salts with inorganic acid, for example, salts with hydrochloric acid, hydrobromic acid, nitric acid, sulfuric acid, phosphoric acid and the like can be mentioned. As preferable examples of the salts with organic acid, for example, salts with formic acid, acetic acid, trifluoroacetic acid, fumaric acid, oxalic acid, tartaric acid, maleic acid, citric acid, succinic acid, malic acid, methanesulfonic acid, benzenesulfonic acid, p-toluenesulfonic acid and the like can be mentioned. As preferable examples of the salts with basic amino acid, for example, salts with arginine, lysine, ornithine and the like can be mentioned, and as preferable examples of the salts with acidic amino acid, for example, salts with aspartic acid, glutamic acid and the like can be mentioned.

As the active ingredient used in the present invention is preferable 2-ethoxy-1-{[2′-(5-oxo-4,5-dihydro-1,2,4-oxadiazol-3-yl)biphenyl-4-yl]methyl}-1H-benzimidazole-7-carboxylic acid (compound A).

As the fat and oil-like substance having a low melting point to be used in the present invention, a fat and oil-like substance generally having a melting point of from about 20° C. to 90° C., preferably from 20° C. to 60° C., is used. Any substance can be used as long as it does not adversely influence the active ingredient. In the production of the pharmaceutical composition of the present invention, the fat and oil-like substance having a low melting point can be added uniformly with the active ingredient as compared to a substances like fat and oil having a high melting point and, as a result, a more stable pharmaceutical composition suppressed decomposition and the like of the active ingredient can be obtained. The fat and oil-like substance having a low melting point may be water-soluble or insoluble. As used herein, examples of water-soluble fat and oil-like substance having a low melting point include the below-mentioned alkylene oxide polymer. As the fat and oil-like substance having a low melting point to be used in the present invention, for example, hydrocarbon, higher fatty acid, higher alcohol, fatty acid ester of polyhydric alcohol, higher alcohol ether of polyhydric alcohol, polymer or copolymer of alkylene oxide and the like can be mentioned, of which fatty acid ester of polyhydric alcohol, higher alcohol ether of polyhydric alcohol, polymer or copolymer of alkylene oxide, particularly, polymer of alkylene oxide, are preferably used.

As hydrocarbon, for example, n-alkane having 17 to 50 carbon atoms such as n-heptadecane, n-octadecane, n-nonadecane, n-eicosane, n-heneicosane, n-docosane, n-tricosane, n-tetracosane, n-pentacosane, n-triacontane, n-pentatriacontane, n-tetracontane, n-pentacontane and the like and mixtures thereof (petrolatum, paraffin wax, microcrystalline wax etc.) and the like can be mentioned.

As the higher fatty acid, for example, capric acid, lauric acid, myristic acid, palmitic acid, stearic acid, arachidonic acid, behenic acid, lignoceric acid, cerotic acid and a mixture thereof, higher fatty acid recovered from natural fat and oil and the like can be mentioned.

As the higher alcohol, for example, lauryl alcohol, myristyl alcohol, cetyl alcohol, stearyl alcohol, arachyl alcohol and a mixture thereof, higher alcohol recovered from natural oil and the like can be mentioned.

As the fatty acid ester of polyhydric alcohol, esters of alcohol having two or more hydroxyl groups in a molecule (e.g., alkylene glycol such as ethylene glycol, propylene glycol and the like, polyalkylene glycols such as polyethylene glycol, polypropylene glycol or copolymers thereof and the like, saccharides such as sorbitol, saccharose and the like, intramolecular dehydrating compound of sorbitol such as 1,5-sorbitan, 1,4-sorbitan, 3,6-sorbitan and the like, glycerol, diethanolamine, pentaerythritol and the like) and fatty acid (e.g., acetic acid, propionic acid, butyric acid, pelargonic acid, capric acid, undecyl acid, lauric acid, tridecyl acid, myristic acid, pentadecyl acid, palmitic acid, heptadecyl acid, stearic acid, nonadecane acid, undecylene acid, oleic acid, elaidic acid, sorbic acid, linolic acid, linolenic acid, arachidonic acid, stearol acid and the like), specifically, for example, sorbitan fatty acid ester having a molecular weight of from 400 to 900 such as sorbitan monostearate, sorbitan tristearate, sorbitan monooleate, sorbitan sesquioleate, sorbitan monopalmitate and the like; polyoxyalkylene sorbitan fatty acid ester having a molecular weight of from 1000 to 1500 such as polyoxyethylene sorbitan tristearate, polyoxyethylene sorbitan monooleate, polyoxyethylene sorbitan tripalmitate and the like; polyoxyalkylene sorbitol fatty acid esters such as polyoxyethylene sorbitol hexastearate, polyoxyethylene sorbitol hexaoleate, polyoxyethylene sorbitol tristearate, polyoxyethylene sorbitol tetralaurate and the like; polyoxyalkylene sorbitol beewax derivatives such as polyoxyethylene sorbitol beewax derivative and the like; polyoxyalkylene lanolin derivatives such as polyoxyethylene lanolin derivative and the like; propylene glycol fatty acid esters having a molecular weight of from 200 to 700 such as propylene glycol monopalmitate, propylene glycol monostearate, propylene glycol dilaurate, propylene glycol dimyristate, propylene glycol dipalmitate, propylene glycol distearate and the like; alkylene glycol fatty acid ester such as ethylene glycol fatty acid esters having a molecular weight of from 500 to 1200 such as ethylene glycol monolaurate, ethylene glycol palmitate, ethylene glycol margarate, ethylene glycol stearate, ethylene glycol dilaurate, ethylene glycol dimyristate, ethylene glycol dipalmitate, ethylene glycol dimargarate and the like; polyoxyalkylene castor oil derivatives having a molecular weight of from 3500 to 4000 such as polyoxyethylene castor oil derivative and the like; polyoxyalkylene fatty acid esters having a molecular weight of from 1900 to 2200 such as polyoxyethylene stearate, polyoxyethylene oleate, polyoxyethylene palmitate, polyoxyethylene linolate and the like; glycerol monofatty acid esters having a molecular weight of from 300 to 600 such as glycerol monoacetate, glycerol monopropionate, glycerol monostearate, glycerol monooleate, glycerol monopalmitate, glycerol monolinolate and the like; sucrose esters of fatty acids having a molecular weight of from 400 to 1300 such as saccharose monolaurate, saccharose monomyristate, saccharose monopalmitate, saccharose monostearate, saccharose trimyristate, saccharose tripalmitate, saccharose tristearate and the like, and the like can be mentioned.

As the higher alcohol ethers of polyhydric alcohol, ethers of polyhydric alcohol (those recited as the alcohol component of the above-mentioned fatty acid ester of polyhydric alcohol) and higher fatty acid alcohol (e.g., cetyl alcohol, stearyl alcohol, oleyl alcohol, octyl alcohol, decyl alcohol), specifically, for example, polyoxyethylene higher alcohol ethers such as polyoxyethylene lauryl alcohol ether, polyoxyethylene cetyl alcohol ether, polyoxyethylene stearyl alcohol ether, polyoxyethylene oleyl alcohol ether, polyoxyethylene octyl alcohol ether, polyoxyethylene decyl alcohol ether and the like, polyoxypropylenepolyoxyethylene higher alcohol ethers such as polyoxypropylenepolyoxyethylene cetyl alcohol ether, polyoxypropylenepolyoxyethylene stearyl alcohol ether, polyoxypropylenepolyoxyethylene oleyl alcohol ether, polyoxypropylenepolyoxyethylene octylalcohol ether, polyoxypropylenepolyoxyethylene lauryl alcohol ether and the like, and the like are frequently used.

As the polymers of alkylene oxide, those having a molecular weight of from 1,000 to 10,000 (e.g., polyethylene glycol 6000 (Macrogol 6000) etc.) is preferably used. As the alkylene oxide, for example, ethylene oxide, propylene oxide, trimethylene oxide, tetrahydrofuran and the like (preferably, ethylene oxide) can be mentioned. As the copolymers of alkylene oxide, a copolymer of two or more from the above-mentioned alkylene oxides and having a molecular weight of from 1,000 to 10,000 is preferably used. These fat and oil-like substances having a low melting point may be used alone or in a combination of two or more kinds thereof.

As the low viscosity binder to be used in the present invention, a binder having a viscosity of less than about 6 mPa·s, preferably about 1-about 6 mPa·s, more preferably about 1-about 4 mPa·s, as measured at 20° C. using a 2% aqueous solution model B viscometer (Brookfield-type viscometer) is used, and any binder is used as long as it does not exert an adverse influence on the active ingredient. As the binder, for example, cellulose derivative, pregelatinized starch, partly pregelatinized starch, polyvinylpyrrolidone, pullulan, dextrin, gum arabic and the like are used, with preference given to cellulose derivatives. As the cellulose derivative, for example, hydroxypropylcellulose, hydroxypropylmethylcellulose, methylcellulose, carboxymethylcellulose and the like are used, with preference given to hydroxypropylcellulose.

As the low viscosity binder to be used in the present invention, hydroxypropylcellulose (e.g., commercially available products such as NIPPON SODA CO., LTD. SSL grade, SL grade and the like) having a viscosity of about 1-about 4 mPa·s as measured at 20° C. using a 2% aqueous solution model B viscometer is preferable.

As the solid pharmaceutical composition of the present invention, for example, a solid dosage form suitable for oral administration such as tablet, granule, fine granules, capsule, pill and the like can be mentioned, with preference given to tablet.

The solid dosage form can be produced by a method known per se (e.g., the method described in the Japanese Pharmacopoeia 14th Edition, General Principles). For example, a tablet can be produced by incorporating a low viscosity binder and a fat and oil-like substance having a lower melting point into the active ingredient, followed by subjecting the mixture to molding. The incorporation is conducted by a method conventionally employed in the field of pharmaceutical preparations, such as mixing, kneading, massing, sieving, stirring and the like. For example, a low viscosity binder, an active ingredient and a fat and oil-like substance having a lower melting point may be directly mixed (addition in a powder state), or a solvent is added to the mixture, followed by conventional kneading, granulating and drying. Alternatively, a fat and oil-like substance having a lower melting point and a low viscosity binder are dissolved in a suitable solvent, then the solution is uniformly mixed with the active ingredient, followed by conventional kneading, granulating and drying (addition in a liquid state). Furthermore, a liquid material containing a low viscosity binder and a fat and oil-like substance having a lower melting point and a liquid material containing the active ingredient can be independently sprayed onto a powder material such as an excipient, followed by mixing the resultant material. In the case of “addition in a liquid state”, any solvent which does not exert undesirable influence on the active ingredient, for example, water, dimethylformamide, acetone, ethanol, propyl alcohol, isopropyl alcohol, butyl alcohol, methylene chloride, trichloroethane etc., can be employed. After completion of blending, the material is subjected to a conventional molding process under pressurization to prepare tablets containing the active ingredient. The molding under pressurization means that a material is compressed under pressurization into a desired form, which most generally refers to tabletting.

It is also possible to add a variety of additives to be employed for preparation making to the solid pharmaceutical composition of the present invention in an adequate step. For example, excipients such as crystalline cellulose (e.g. Avicel PH 101 (manufactured by Asahi Chemical Industry Co., Ltd.)), carboxymethyl cellulose calcium, corn starch, wheat starch, lactose, sucrose, glucose, calcium sulfate, calcium phosphate, sodium chloride etc., binders such as gum arabic, gelatin, methyl cellulose, polyvinyl pyrrolidone, hydroxypropyl cellulose (hereinafter sometimes abbreviated as HPC), hydroxypropylmethyl cellulose etc., lubricants such as magnesium stearate, talc, synthetic aluminum silicate, sodium lauryl sulfate, boric acid, magnesium oxide, paraffin etc., colorants, flavoring agents, odor-improving agents, and the like may be added.

Furthermore, the solid pharmaceutical composition of the present invention can also be prepared into coated tablets. The coating may be conducted by a method known per se. As the coating agents, conventional coating agents (e.g. hydroxypropylmethyl cellulose, hydroxypropyl cellulose, methyl cellulose, polyvinyl pyrrolidone etc.), and as auxiliary agents for coating, use is made of, for example, polyethylene glycol 6000, polysorbate (e.g. Tween 80 etc.), titanium oxide, and pigments such as red iron oxide can be used.

The solid pharmaceutical composition of the present invention contains a low viscosity binder in a proportion of (coated tablet is without coating) 0.5-15 wt %, preferably 1-10 wt %, more preferably 2-5 wt %, in the composition. The fat and oil-like substance having a low melting point is contained in a proportion of (coated tablet is without coating) 0.5-15 wt %, preferably 1-10 wt %, more preferably 2-5 wt %, in the composition. The active ingredient is contained in a proportion of (coated tablet is without coating) 0.1-40 wt %, preferably 1-30 wt %, more preferably 2-25 wt %, in the composition. The content of the active ingredient is about 1-about 150 mg, preferably about 2-about 100 mg, more preferably about 2-about 80 mg.

From the aspect of disintegratability, the solid pharmaceutical composition of the present invention preferably disintegrates within 30 min in an aqueous solution. The solid pharmaceutical composition of the present invention thus-obtained by adding a fat and oil-like substance having a low melting point and a low viscosity binder to the active ingredient suppresses decomposition with time due to molding and becomes a clinically extremely useful preparation superior in the dissolution property.

The solid pharmaceutical composition of the present invention can be safely administered as a pharmaceutical agent for a mammal (e.g., human, dog, rabbit, rat, mouse and the like).

The dose of a particular patient is determined in consideration of the age, body weight, general health condition, sex, diet, administration time, clearance rate, drug combination and the like, as well as the severity of the disease for which the patient is undergoing the treatment. The daily dose is about 0.05-500 mg, preferably 0.1-100 mg, as a compound represented by the formula (I).

As the “low viscosity binder”, “active ingredient” and “fat and oil-like substance having a low melting point” in the “method of improving dissolution of an active ingredient from a solid pharmaceutical composition comprising the active ingredient and a fat and oil-like substance having a low melting point, which comprises using a low viscosity binder” of the present invention, those mentioned above and the like can be mentioned. As the “solid pharmaceutical composition”, those exemplified as the above-mentioned solid pharmaceutical composition of the present invention and the like can be mentioned. According to the method of the present invention, for example, the dissolution of the active ingredient from a solid pharmaceutical composition can be improved by adding a low viscosity binder to a solid pharmaceutical composition containing the active ingredient and a fat and oil-like substance having a low melting point.

### EXAMPLES

The present invention is explained in more detail in the following by referring to Examples and Reference Examples, which are not to be construed as limitative.

Compound A is 2-ethoxy-1-{[2′-(5-oxo-4,5-dihydro-1,2,4-oxadiazol-3-yl)biphenyl-4-yl]methyl}-1H-benzimidazole-7-carboxylic acid (melting point: 191° C., solubility in water at 20° C. of about 0.006 g/L). In the following Examples and Reference Examples, the Japanese Pharmacopoeia 14th Edition or Japanese Pharmaceutical Excipients 2003 compatible products were used as the preparation additives. Of the preparation additives, while magnesium stearate is also the Japanese Pharmacopoeia 14th Edition compatible product, like other preparation additives, it particularly has a stearic acid content ratio of not less than about 90% (Taihei Chemical Industrial Co., Ltd.).

### Example 1

Using a fluidized bed granulator (POWREX, Lab-1) and according to the following formulation (Table 1), compound A obtained in Reference Example 4, lactose and cornstarch were mixed, and an aqueous solution of polyethylene glycol 6000 as a fat and oil-like substance having a low melting point in hydroxypropylcellulose (viscosity 2-3.4 mPa·s) was sprayed as a binder liquid, granulated, dried and sized. Low-substituted hydroxypropylcellulose and magnesium stearate were added and mixed, and the mixture was tabletted using a tabletting machine (Shimadzu Corporation, AUTOGRAPH AG-1) with a 8.0 mmφ biconvex punch at weight 200 mg, pressure 8.5 kN.

### Example 2

Using a fluidized bed granulator (POWREX, Lab-1) and according to the following formulation (Table 1), compound A obtained in Reference Example 4, lactose and cornstarch were mixed, and an aqueous solution of polyethylene glycol 6000 as a fat and oil-like substance having a low melting point in hydroxypropylcellulose (viscosity 2-3.4 mPa·s) was sprayed as a binder liquid, granulated, dried and sized. Low-substituted hydroxypropylcellulose and magnesium stearate were added and mixed, and the mixture was tabletted using a tabletting machine (Shimadzu Corporation, AUTOGRAPH AG-1) with a 13 mm×8 mm oval type convex punch at weight 400 mg, pressure 10.5 kN.

### Example 3

Using a fluidized bed granulator (POWREX, FD-5S) and according to the following formulation (Table 2), compound A obtained in Reference Example 5, lactose and cornstarch were mixed, and an aqueous solution of polyethylene glycol 6000 as a fat and oil-like substance having a low melting point in hydroxypropylcellulose (viscosity 2-3.4 mPa·s) was sprayed as a binder liquid, granulated, dried and sized. Low-substituted hydroxypropylcellulose, crystalline cellulose and magnesium stearate were added and mixed, and the mixture was tabletted using a tabletting machine (KIKUSUI SEISAKUSHO LTD., Correct 19K) with a 7 mmφ biconvex punch at weight 130 mg, pressure 7 kN.

### Example 4

Using a fluidized bed granulator (POWREX, FD-5S) and according to the following formulation (Table 2), compound A obtained in Reference Example 5, lactose and cornstarch were mixed, and an aqueous solution of polyethylene glycol 6000 as a fat and oil-like substance having a low melting point in hydroxypropylcellulose (viscosity 2-3.4 mPa·s) was sprayed as a binder liquid, granulated, dried and sized. Low-substituted hydroxypropylcellulose, crystalline cellulose and magnesium stearate were added and mixed, and the mixture was tabletted using a tabletting machine (KIKUSUI SEISAKUSHO LTD., Correct 19K) with a 8.5 mmφ biconvex punch at weight 260 mg, pressure 8 kN.

### Example 5

Using a fluidized bed granulator (POWREX, FD-5S) and according to the following formulation (Table 2), compound A obtained in Reference Example 5, lactose, cornstarch and crystalline cellulose were mixed, and an aqueous solution of polyethylene glycol 6000 as a fat and oil-like substance having a low melting point in hydroxypropylcellulose (viscosity 2-3.4 mPa·s) was sprayed as a binder liquid, granulated, dried and sized. Low-substituted hydroxypropylcellulose, crystalline cellulose and magnesium stearate were added and mixed, and the mixture was tabletted using a tabletting machine (KIKUSUI SEISAKUSHO LTD., Correct 19K) with a 7 mmφ biconvex punch at weight 130 mg, pressure 7 kN.

### Example 6

Using a fluidized bed granulator (POWREX, FD-5S) and according to the following formulation (Table 2), compound A obtained in Reference Example 5, lactose, cornstarch and crystalline cellulose were mixed, and an aqueous solution of polyethylene glycol 6000 as a fat and oil-like substance having a low melting point in hydroxypropylcellulose (viscosity 2-3.4 mPa·s) was sprayed as a binder liquid, granulated, dried and sized. Low-substituted hydroxypropylcellulose, crystalline cellulose and magnesium stearate were added and mixed, and the mixture was tabletted using a tabletting machine (KIKUSUI SEISAKUSHO LTD., Correct 19K) with a 8.5 mmφ biconvex punch at weight 260 mg, pressure 7 kN.

### Reference Example 1

Using a fluidized bed granulator (POWREX, FD-5S) and according to the following formulation, compound A obtained in Reference Example 4, lactose and cornstarch were mixed, and an aqueous solution of polyethylene glycol 6000 as a fat and oil-like substance having a low melting point in hydroxypropylcellulose (viscosity 6-10 mPa·s) was sprayed as a binder liquid, granulated, dried and sized. Low-substituted hydroxypropylcellulose and magnesium stearate were added and mixed, and the mixture was tabletted using a tabletting machine (KIKUSUI SEISAKUSHO LTD., Correct 19K) with a 6.5 mmφ biconvex punch at weight 100 mg, pressure 7 kN.

### Reference Example 2

Using a fluidized bed granulator (POWREX, Lab-1) and according to the following formulation, compound A obtained in Reference Example 4, lactose and cornstarch were mixed, and an aqueous solution of polyethylene glycol 6000 as a fat and oil-like substance having a low melting point in hydroxypropylcellulose (viscosity 6-10 mPa·s) was sprayed as a binder liquid, granulated, dried and sized. Low-substituted hydroxypropylcellulose and magnesium stearate were added and mixed, and the mixture was tabletted using a tabletting machine (Shimadzu Corporation, AUTOGRAPH AG-1) with a 8.0 mmφ biconvex punch at weight 200 mg, pressure 8.5 kN.

### Reference Example 3

Using a fluidized bed granulator (POWREX, Lab-1) and according to the following formulation, compound A obtained in Reference Example 4, lactose and cornstarch were mixed, and an aqueous solution of polyethylene glycol 6000 as a fat and oil-like substance having a low melting point in hydroxypropylcellulose (viscosity 6-10 mPa·s) was sprayed as a binder liquid, granulated, dried and sized. Low-substituted hydroxypropylcellulose and magnesium stearate were added and mixed, and the mixture was tabletted using a tabletting machine (Shimadzu Corporation, AUTOGRAPH AG-1) with a 13 mm×8 mm oval type convex punch at weight 400 mg, pressure 10.5 kN.

### Reference Example 4

To methyl 2-ethoxy-1-{[2′-(5-oxo-4,5-dihydro-1,2,4-oxadiazol-3-yl)biphenyl-4-yl]methyl}-1H-benzimidazole-7-carboxylate (10 g) was added 0.40N-NaOH (167 mL) and the mixture was stirred at 65-75° C. for 1-1.5 hr. The mixture was adjusted to pH 8 at room temperature with 1N HCl, activated carbon (0.5 g) was added and the mixture was stirred. The activated carbon was filtered off and the residue was washed with water (17 mL). The mixture was adjusted to pH 3 with 1N HCl at 0-5° C. The mixture was stirred at 40-45° C. and then at 0-10° C. The precipitated crystals were collected by filtration, washed with water (17 mL×2 times), and dried at 40° C. to give compound A as a white powder (9.3 g, yield 96%).

### Reference Example 5

To methyl 2-ethoxy-1-{[2′-(5-oxo-4,5-dihydro-1,2,4-oxadiazol-3-yl)biphenyl-4-yl]methyl}-1H-benzimidazole-7-carboxylate (10 g) was added 0.36N-NaOH (150 mL) and the mixture was stirred at 65-75° C. for 1.5 hr. The mixture was adjusted to pH 8 at room temperature with 1N HCl, activated carbon (0.5 g) was added and the mixture was stirred. The activated carbon was filtered off and the residue was washed with water (50 mL). The mixture was adjusted to pH 3 with 0.5N HCl at 9-15° C. The mixture was stirred at 40-45° C. and then at 5-15° C. The precipitated crystals were collected by filtration, washed with water (20 mL), and dried at 40° C. to give compound A as a white powder (9.3 g, yield 96%).

### Experimental Example

The tablets obtained in Examples 1, 2 and Reference Examples 1, 2, 3 were subjected to a test according to the Dissolution Test Method 2 (Paddle Method, 50 rpm, 37° C.) and using phosphate buffer, pH 6.8/water mixture (1:1), 900 mL, as a test solution.

TABLE 1

| | Ref. | Ref. | Ref. | | |
|---|---|---|---|---|---|
| composition | Ex. 1 | Ex. 2 | Ex. 3 | Ex. 1 | Ex. 2 |
| compound A | 10.0 | 20.0 | 40.0 | 20.0 | 40.0 |
| lactose | 51.0 | 102.0 | 204.0 | 102.0 | 204.0 |
| cornstarch | 23.0 | 46.0 | 92.0 | 46.0 | 92.0 |
| hydroxypropylcellulose | 3.0 | 6.0 | 12.0 | — | — |
| (viscosity 6-10 mPa · s) | | | | | |
| hydroxypropylcellulose | — | — | — | 6.0 | 12.0 |
| (viscosity 2-3.4 mPa · s) | | | | | |
| macrogol 6000 | 3.0 | 6.0 | 12.0 | 6.0 | 12.0 |
| low-substituted | 9.5 | 19.0 | 38.0 | 19.0 | 38.0 |
| hydroxypropylcellulose | | | | | |
| magnesium stearate | 0.5 | 1.0 | 2.0 | 1.0 | 2.0 |
| total | 100.0 | 200.0 | 400.0 | 200.0 | 400.0 |

TABLE 2

| composition | Ex. 3 | Ex. 4 | Ex. 5 | Ex. 6 |
|---|---|---|---|---|
| compound A | 20 | 40 | 40 | 80 |
| lactose | 53.9 | 107.8 | 29.3 | 58.6 |
| cornstarch | 20 | 40 | 13 | 26 |
| hydroxypropylcellulose | 4 | 8 | 4 | 8 |
| (viscosity 2-3.4 mPa · s) | | | | |
| macrogol 6000 | 4 | 8 | 4 | 8 |
| low-substituted | 12.4 | 24.8 | 13 | 26 |
| hydroxypropylcellulose | | | | |
| crystalline cellulose | 15 | 30 | 26 | 52 |
| magnesium stearate | 0.7 | 1.4 | 0.7 | 1.4 |
| total | 130 | 260 | 130 | 260 |
| shape | 7 mmφ | 8.5 mmφ | 7 mmφ | 8.5 mmφ |

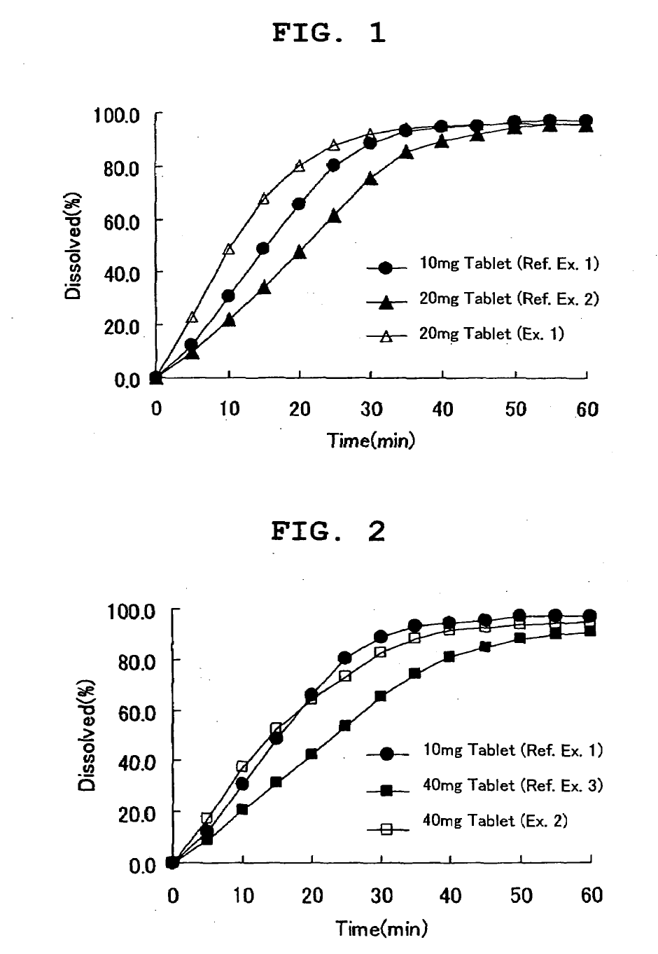

As shown in FIGS. 1 and 2, a tablet containing a low viscosity binder shows superior dissolution property as compared with a tablet containing a binder having a conventional viscosity, and by the addition of a low viscosity binder, the drug dissolution property could be easily controlled.

### INDUSTRIAL APPLICABILITY

Since the solid pharmaceutical composition of the present invention is simultaneously superior in the stability and dissolution property, it is extremely useful as a pharmaceutical product preparation technique.

This application is based on application No. 2006-218145 filed in Japan, the contents of which are incorporated hereinto by reference.

1. A solid pharmaceutical composition comprising an active ingredient, a fat and oil-like substance having a low melting point and a low viscosity binder.

2. The composition of claim 1, wherein the active ingredient is a crystalline poorly-soluble compound.

3. The composition of claim 2, wherein the crystalline poorly-soluble compound has a melting point of about 75° C.-about 250° C., and water solubility of not more than about 1 g/L.

4. The composition of claim 2, wherein the crystalline poorly-soluble compound is a compound represented by the formula (I): 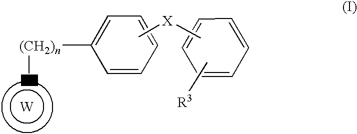
wherein the ring W is an optionally substituted N-containing heterocyclic residue; R³ is a group capable of forming anion or a group convertible thereto; X is a direct bond or a spacer having an atomic length of two or less between the phenylene group and the phenyl group; and n is an integer of 1 or 2, or a salt thereof.

5. The composition of claim 4, wherein the compound represented by the formula (I) or a salt thereof is 2-ethoxy-1-{[2′-(5-oxo-4,5-dihydro-1,2,4-oxadiazol-3-yl)biphenyl-4-yl]methyl}-1H-benzimidazole-7-carboxylic acid.

6. The composition of claim 1, wherein the fat and oil-like substance has a melting point of 20° C.-90° C.

7. The composition of claim 1, wherein the low viscosity binder is a low viscosity cellulose derivative.

8. The composition of claim 7, wherein the cellulose derivative is hydroxypropylcellulose.

9. The composition of claim 1, wherein the low viscosity binder is hydroxypropylcellulose having a viscosity of about 1-about 4 mPa·s.

10. The composition of claim 1, which is a tablet.

11. A method of improving dissolution of an active ingredient from a solid pharmaceutical composition comprising the active ingredient and a fat and oil-like substance having a low melting point, which comprises using a low viscosity binder.
